(19) United States
(12) Patent Application Publication
(10) Pub. No.: US 2011/0178571 A1
(43) Pub. Date: Jul. 21, 2011

(54) Method and Apparatus for Nerve and Muscle Stimulation and Pain Treatment

(75) Inventors: Joseph Tannebaum, Jerusalem (IL); Hedva Romanoff, Sylvania, OH (US); Aviva Scheer, Sarasota, FL (US); Eli Shavit Pasternak, Holon (IL)

(73) Assignee: PAINLESS MEDICAL TECHNOLOGIES, LTD., Tel Aviv (IL)

(21) Appl. No.: 12/595,960

(22) PCT Filed: Apr. 27, 2008
(86) PCT No.: PCT/IL08/00557

§ 371 Date: Aug. 16, 2010

Related U.S. Application Data
(60) Provisional application No. 60/912,797, filed on Apr. 19, 2007.

(51) Int. Cl. A61N 1/36

(52) U.S. Cl. 607/46

(57) ABSTRACT

An apparatus for transcutaneous stimulation comprising: a pulse generator operative to generate repetitive pulses exhibiting a pulse width of 25-60 microseconds, a consistent pulse rise time of no more than 5% of the pulse width and an inter-pulse interval of between 0.1 and 3 milliseconds; an intra-group modulator producing modulated pulses exhibiting an amplitude of between 50% and 100% of a maximum modulated pulse amplitude in a generally increasing manner, the modulated pulses defining a group of pulses, the intra-group modulator being further operative to modulate the pulses to exhibit an amplitude of no more than 25% of the maximum modulated pulse amplitude for a predetermined time period between successive groups of pulses thereby creating a pulse train; and an output modulator modulating the pulse train to produce output pulses exhibiting an amplitude of between 50% and 100% of a maximum according to a predetermined repetitive waveform.

### BACKGROUND OF THE INVENTION

The invention relates generally to the field of pain relief devices and more particularly to a device for nerve blocking and/or muscle stimulation through transcutaneous application of electric current.

Transcutaneous electric nerve stimulation (TENS) for pain control has been in use for many years; however medical science remains skeptical of the ability of TENS to block pain in excess of the placebo effect. Few carefully controlled studies have been performed on TENS over the years, and those have generally not found TENS to be ameliorative. However, inadequately randomized studies consistently report that TENS is effective, and TENS remains in broad use throughout the world.

Pain signals reach the brain via nerves and the spinal cord. TENS is thought to either affect the way pain signals are sent to the brain by blocking the transmission function of the nerve or by distracting the brain from the pain signal. If pain signals can be blocked then the brain will receive fewer signals from the source of the pain, and the patient may thus feel less pain. TENS is applied either in a high frequency mode, in which a high pulse rate is thought to trigger a pain gate to close thereby blocking the nerve pathway to the brain; or in a low frequency mode of around 2-5 hertz which is thought to stimulate the patient body to make its own pain easing chemicals called endorphins which act to block pain signals. By far, the high frequency mode is more prevalent and believed to be more effective.

Unfortunately, as indicated above, TENS has not yet been successfully proven to ameliorate pain consistently in well designed randomized trials. The inventors believe that this is in part due to the inappropriate waveforms being utilized by the prior art, which are unable to penetrate large myelinated fibers, and block the pathway to the brain.

There is thus a long felt need for an improved method and apparatus for transcutaneous nerve blocking, and in particular one whose waveforms are effective in ameliorating pain.

### SUMMARY OF THE INVENTION

In certain embodiments the invention provides for an apparatus operative to apply modulated pulses of short duration to the area to be treated. In one particular embodiment the area to be treated comprises two points preferably at least 4 centimeters apart generally in consonance with the nerve to be blocked and the modulated pulses comprise constant current pulses of approximately 100 mA each. The pulses exhibit a varying amplitude whose maximum is preferably approximately 100V and are preferably applied to about a 16 mm² skin patch.

In a preferred embodiment the pulses are of a constant width, preferably of 25-60 microseconds, even further preferably 25-50 microseconds, with a rise and fall time of no more than 5% of the pulse width, with an inter-pulse interval of between 0.1 and 3 milliseconds.

In certain embodiment the pulses are modulated to exhibit a generally increasing amplitude between 50%-100% of a modulated pulse amplitude, preferably 70%-100%. The modulated pulses are arranged in pulse groups, with an amplitude between groups of no more than 25% of the modulated pulse amplitude, and preferably approximately 0% of the modulated pulse amplitude, thereby creating a pulse train. The intra-group modulation preferably gradually increases the pulse amplitude over a predetermined time period of between 5 and 25 milliseconds, and preferably on the order of 10 milliseconds.

In one embodiment the inter-group time period is between 10-200 milliseconds.

The modulated groups of pulses are further preferably output modulated to exhibit an output amplitude of between 50%-100% of a maximum amplitude by one of a triangular waveform and a deltoid waveform. Preferably the output modulation exhibits a period of 3-5 seconds. In one embodiment the pulse train is modulated with a deltoid waveform, with the rise and fall of the deltoid modulation preferably being each approximately ⅓ of the total deltoid period and a steady state portion of the deltoid waveform exhibiting a period of approximately ⅓ of the total deltoid period. In one particular preferred embodiment the deltoid modulation waveform exhibits a generally increasing linear slope for about 1 second, a generally unchanged maximum output for about 1½ second and a generally decreasing slope for about 1 second for a total period of about 3½ seconds.

In another embodiment the pulse train is modulated with a triangular waveform, with the rise and fall of the triangular modulation preferably being of equal duration. The particular pulses and modulation thereof is successful in providing improved pain relief.

In yet another embodiment the pulses are directly output modulated with one of a triangular waveform and a deltoid waveform. In yet another embodiment the pulse train is output without further modulation.

In certain embodiments the apparatus provides for a regional anesthesia. Advantageously, in some particular embodiments the apparatus allows for more robust muscle stimulation without undue pain. In certain embodiment the apparatus provides for simultaneous pain relief and muscle stimulation.

Additional features and advantages of the invention will become apparent from the following drawings and description.

### BRIEF DESCRIPTION OF THE DRAWINGS

For a better understanding of the invention and to show how the same may be carried into effect, reference will now be made, purely by way of example, to the accompanying drawings in which like numerals designate corresponding elements or sections throughout.

With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice. In the accompanying drawings:

FIG. 1 illustrates a high level schematic diagram of an embodiment of a modulated pulse generator in accordance with certain embodiments of the invention;

FIG. 2A illustrates the output of the pulse generator of FIG. 1 in accordance with certain embodiments of the invention;

FIG. 2B illustrates the intra-group modulation and the inter-group modulation of the repetitive pulses of FIG. 2A defining a pulse train in accordance with certain embodiments of the invention;

FIG. 2C illustrates a triangular modulation envelope for the pulse train of FIG. 2B in accordance with certain embodiments of the invention;

FIG. 2D illustrates a deltoid modulation envelope for the pulse train of FIG. 2B in accordance with certain embodiments of the invention; and

FIGS. 3-5 illustrate high level flow chart of the operation of the modulated pulse generator of FIG. 1 in accordance with certain embodiments of the invention.

### DETAILED DESCRIPTION OF THE PREFERRED EMBODIMENTS

The present embodiments enable an apparatus operative to apply modulated pulses of short duration to the area to be treated. In an exemplary embodiment the area to be treated comprises two points preferably at least 4 centimeters apart generally in consonance with the nerve to be blocked and the modulated pulses comprise constant current pulses of approximately 100 mA each. The pulses exhibit a varying amplitude whose maximum is preferably approximately 100V and are preferably applied to a 16 mm² skin patch.

In a preferred embodiment the pulses are of a constant width, preferably of 25-60 microseconds, even further preferably 25-50 microseconds, with a rise and fall time of no more than 5% of the pulse width, with an inter-pulse interval of between 0.1 and 3 milliseconds.

In certain embodiments the pulses are modulated to exhibit a generally increasing amplitude between 50%-100% of a modulated pulse amplitude, preferably 70%-100%. The modulated pulses are arranged in pulse groups, with an amplitude between groups of no more than 25% of the modulated pulse amplitude, and preferably approximately 0% of the modulated pulse amplitude, thereby creating a pulse train. The intra-group modulation preferably gradually increases the pulse amplitude over a predetermined time period of between 5 and 25 milliseconds, and preferably on the order of 10 milliseconds.

In one embodiment the inter-group time period is between 10-200 milliseconds.

In certain embodiments the modulated groups of pulses are further preferably output modulated to exhibit an output amplitude of between 50%-100% of a maximum amplitude by one of a triangular waveform and a deltoid waveform. Preferably, the maximum amplitude is user selectable. Preferably the output modulation exhibits a period of 3-5 seconds. In one embodiment the pulse train is modulated with a deltoid waveform, with the rise and fall of the deltoid modulation preferably being each approximately ⅓ of the total deltoid period and a steady state portion of the deltoid waveform exhibiting a period of approximately ⅓ of the total deltoid period. In one particular preferred embodiment the deltoid modulation waveform exhibits a generally increasing linear slope for about 1 second, a generally unchanged maximum output for about 1½ second and a generally decreasing slope for about 1 second for a total period of about 3½ seconds.

In another embodiment the pulse train is modulated with a triangular waveform, with the rise and fall of the triangular modulation preferably being of equal duration. The particular pulses and modulation thereof is successful in providing improved pain relief.

In certain embodiments the apparatus, exhibiting the particular pulses and modulation thereof, provides for a regional anesthesia. Advantageously, in some particular embodiments the apparatus allows for more robust muscle stimulation without undue pain. In certain embodiment the apparatus thus provides for simultaneous pain relief and muscle stimulation.

Before explaining at least one embodiment of the invention in detail, it is to be understood that the invention is not limited in its application to the details of construction and the arrangement of the components set forth in the following description or illustrated in the drawings. The invention is applicable to other embodiments or of being practiced or carried out in various ways. Also, it is to be understood that the phraseology and terminology employed herein is for the purpose of description and should not be regarded as limiting.

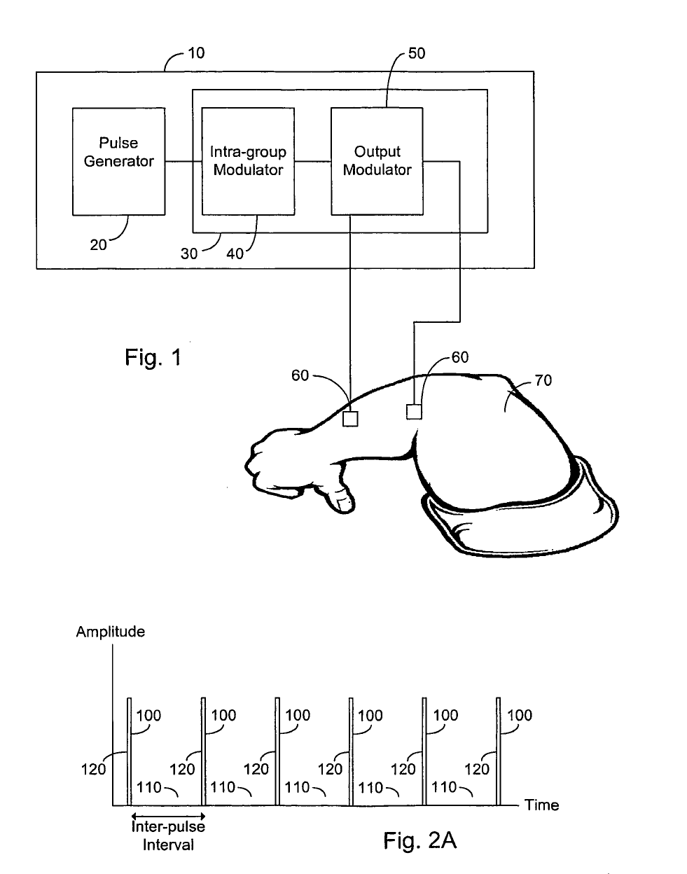

FIG. 1 illustrates a high level schematic diagram of an embodiment of a modulated pulse generator 10, in accordance with certain embodiments of the invention, comprising: a pulse generator 20; and a modulator 30 comprising an intra-group modulator 40 and an output modulator 50. The outputs of output modulator 50 are shown connected to a pair of applicators 60 attached transcutaneously to a patient 70 preferably exhibiting a distance of at least 4 cm between applicators 60. In a preferred embodiment applicators 60 are placed generally along the length of a nerve whose pain transmission is to be blocked. Further preferably the nerve is a large myelinated nerve.

The output of pulse generator 20 is connected to the input of intra-group modulator 40 of modulator 30. The output of intra-group modulator 40 is connected to the input of output modulator 50. Intra-group modulator 40 is shown as being separate from output modulator 50, however this is not meant to be limiting in any way. In one embodiment pulse generator 20, intra-group modulator 40 and output modulator 50 are accomplished in a single micro-controller without exceeding the scope of the invention. Such a micro-controller implementation preferably comprises a digital to analog converter, either internally or externally, arranged to output the modulated pulse waveform. Output modulator 50 preferably further comprises a constant current driver without exceeding the scope of the invention, the intensity of the driver preferably being user selectable to define the maximum output amplitude. In an embodiment in which a micro-controller is used, such a constant current driver is in one particular embodiment external to the micro-controller, and coupled to the output thereof.

It is to be understood that in certain embodiments only one of intra-group modulator 40 and output modulator 50 are required. Additionally, random pulses may further injected, as taught by U.S. Pat. No. 4,977,895 issued Dec. 18, 1990 to Tannenbaum, the entire contents of which is incorporated herein by reference.

In operation, pulse generator 20 generates repetitive pulses with a width of 25-60 microseconds, a consistent pulse rise time of no more than 5% of the pulse width and an inter-pulse interval of between 0.1 and 3 milliseconds. The pulse width and rise time referred to are defined at the output of modulator 30, and thus the rise time of the pulses is preferably a function of the delivering electronics at the output of output modulator 50. A sharp rise time is preferred; however there is a limitation on rise time due to the inherent capacitance of patient 70. In a preferred embodiment output modulator 50 comprises a controlled current source exhibiting a high rise time.

In one embodiment the pulse width is 25-50 microseconds. In another embodiment the consistent pulse rise time is no more than 4% of the pulse width. In yet another embodiment the consistent pulse rise time is no more than 3% of the pulse width. In yet another embodiment the consistent pulse rise time is approximately 2.5-3 microseconds. In one embodiment the interval between the start time of successive pulses, known as the inter-pulse interval, is between 0.5 and 2 milliseconds. In one particular preferred embodiment the repetitive pulses exhibit a pulse width of about 50 microseconds, a consistent pulse rise time of about 1 microsecond and an inter-pulse interval of between 0.1 and 3 milliseconds.

Intra-group modulator 40 receives the output of pulse generator 20, and modulates the pulses to exhibit a generally rising amplitude between 50%-100% of a modulated pulse maximum, over a pre-determined intra-group period. The pulses exhibiting the generally rising amplitude are referred to herein as a group of pulses.

At the end of the intra-group period, intra-group modulator 50 further modulates the pulses to exhibit an amplitude of no more than 25% of the modulated pulse maximum for a pre-determined inter-group time period. Groups of pulses exhibiting inter-group modulation are known herein as a pulse train.

In one embodiment the generally rising amplitude is between 70%-100% of the modulated pulse maximum. In yet another embodiment the generally rising amplitude is between 80%-100% of the modulated pulse maximum.

In one embodiment the pre-determined intra-group period is 5-25 milliseconds, preferably on the order of 10 milliseconds. In another embodiment the intra-group modulator 40 modulates the pulses to exhibit an amplitude of approximately 0% of the modulated pulse maximum during the pre-determined inter-group time period. In one embodiment the inter-group time period is between 5-200 milliseconds, and in another embodiment the inter-group time period is between 10-200 milliseconds.

Output modulator 50 receives the modulated output of intra-group modulator 40, and further modulates the output according to a pre-determined repetitive waveform. In one embodiment the pre-determined repetitive waveform exhibits a modulated amplitude of 50%-100% of the maximum output amplitude. In a preferred embodiment the pre-determined repetitive waveform exhibits a modulated amplitude of 70%-100% of the maximum output amplitude.

In one embodiment the repetitive waveform is a generally triangular waveform, and in another embodiment the repetitive waveform is a generally deltoid waveform. In one embodiment the repetitive waveform exhibits a period of 3-5 seconds, preferably one of 3 seconds, 4 seconds and 5 seconds. In an embodiment in which a generally triangular waveform is implemented, preferably the waveform exhibits a substantially linear increase and decrease in amplitude, with the increase and decrease being substantially of the same rate of change. In an embodiment in which a generally deltoid waveform is implemented, preferably the output waveform exhibits a generally linear increase in amplitude for approximately ⅓ of the period, a maximum output pulse amplitude of ⅓ of the period and a generally linear decrease in amplitude for approximately ⅓ of the period.

In one embodiment intra-group modulator 50 is not implemented, and output modulator 50 direct modulates the pulses. Preferably the modulation is in accordance with one of the deltoid and triangular waveforms described above.

In one embodiment the modulation functionality of output modulator 50 is not implemented, and the pulse train of intra-group modulator 40 is directly output to the driving section of output modulator 50.

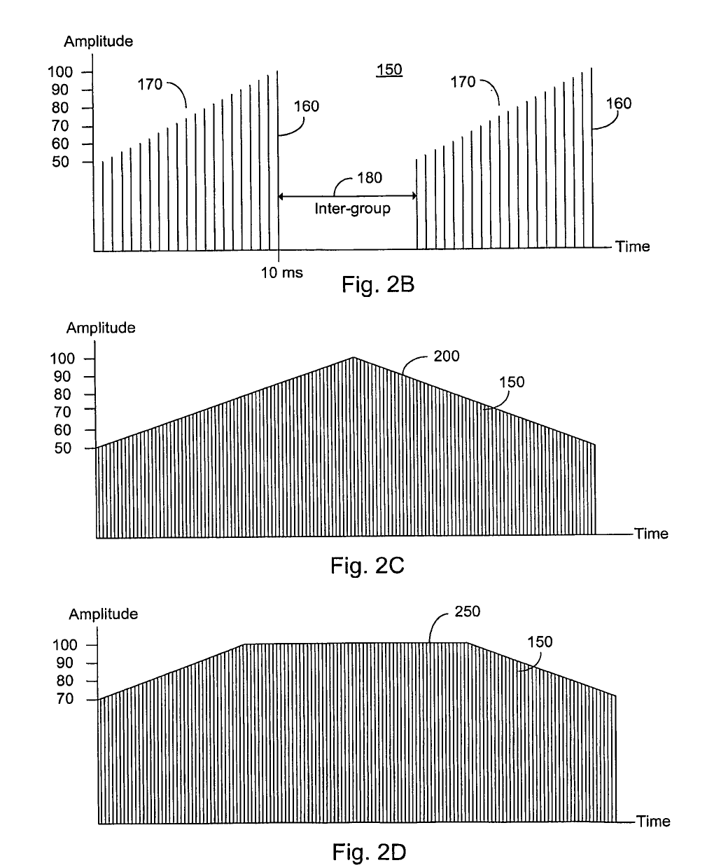

FIG. 2A illustrates the output of pulse generator 20 of FIG. 1 in accordance with certain embodiments, in which the x-axis represents time and the y-axis represents amplitude at the output of output modulator 50. The output of pulse generator 20 exhibits a repetitive pulse 100 each of a width of 25-60 microseconds, preferably 25-50 microseconds, with an inter-pulse interval 110 of 0.1-3 milliseconds, preferably 0.5-2 milliseconds. A sharp rise time 120 is shown for each pulse 100, of no more than 5% of the pulse width. Preferably rise time 120 is no more than 3% of the pulse width. Further preferably rise time 120 is in the range of 2.5-3 microseconds.

FIG. 2B illustrates the intra-group modulation and the inter-group modulation of the repetitive pulses of FIG. 2A defining a pulse train 150 in accordance with certain embodiments of the invention, in which the x-axis represents time and the y-axis represents amplitude at the output of intra-group modulator 40. Pulse train 150 comprises a plurality of pulse groups 160 each separated by an inter-group period 180.

Pulse groups 160 each exhibit a generally increasing amplitude 170. In one embodiment the generally increasing amplitude is linear. Pulse groups 160 are each shown increasing linearly from 50%-100% of the maximum modulated pulse amplitude, however this is not meant to be limiting in any way. In another embodiment, pulse groups 160 each increase from 70%-100%. In yet another embodiment pulse groups 160 each increase from 80%-100%. Pulse groups 160 are shown as increasing over time, however this is not meant to be limiting in any way, and pulse groups 160 may exhibiting a leveling off without exceeding the scope of the invention. In one particular embodiment, pulse groups 160 exhibiting a leveling off period at maximum amplitude of a time duration on the order of 50% of the total time duration of a pulse group 160.

Inter-group period 180 is shown exhibiting approximately 0% of the maximum modulated pulse amplitude; however this is not meant to be limiting in any way. In another embodiment inter-group 180 exhibits an amplitude of no more than 25% of the maximum modulated pulse amplitude without exceeding the scope of the invention.

Inter-group period 180 is preferably between 5-200 milliseconds, and further preferably between 10-200 milliseconds. In one embodiment no inter-group period 180 is provided, and pulse groups 160 are contiguous.

Pulse train 150 thus comprises groups of pulses 160 exhibiting a generally increasing amplitude and an inter-group period exhibiting an amplitude of no more than 25% of the maximum amplitude.

FIG. 2C illustrates a triangular modulation envelope 200 for pulse train 150 of FIG. 2B in accordance with certain embodiments of the invention. Triangular envelope 200 is generated by output modulator 50 and is applied to pulse train 150 output by intra-group modulator 40. Triangular modulation envelope 200 exhibits a regular period, preferably of 3-5 seconds, further preferably one of approximately 3, 4 and 5 seconds. In one embodiment triangular modulation envelope 200 exhibits a generally increasing linear slope for ½ of the period and a generally decreasing linear slope for ½ of the period. In a preferred embodiment the increasing slope and the decreasing slope are of the same absolute value.

Triangular modulation envelope 200 is shown modulating pulse train 150 of FIG. 2B between 50%-100% of the maximum output pulse amplitude; however this is not meant to be limiting in any way. In another embodiment triangular modulation envelope 200 modulates pulse train 150 to exhibit 70%-100% of the maximum output pulse amplitude.

FIG. 2D illustrates a deltoid modulation envelope 250 for pulse train 150 of FIG. 2B in accordance with certain embodiments of the invention. Deltoid envelope 250 is generated by output modulator 50 and is applied to pulse train 150 output by intra-group modulator 40. Deltoid modulation envelope 250 exhibits a regular period, preferably of 3-5 seconds, further preferably one of approximately 3, 4 and 5 seconds. In one embodiment deltoid modulation envelope 250 exhibits a generally increasing linear slope for about ⅓ of the period, a generally unchanged maximum output for about ⅓ of the period and a generally decreasing linear slope for about ⅓ of the period. In a preferred embodiment the increasing slope and the decreasing slope are of the same absolute value. In one particular preferred embodiment deltoid modulation envelope 250 exhibits a generally increasing linear slope for about 1 second, a generally unchanged maximum output for about 1½ second and a generally decreasing slope for about 1 second for a total period of about 3½ seconds.

Deltoid modulation envelope 250 is shown modulating pulse train 150 between 70%-100% of the maximum output pulse amplitude; however this is not meant to be limiting in any way. In another embodiment deltoid modulation envelope 250 modulates pulse train 150 to exhibit 50%-100% of the maximum output pulse amplitude. Advantageously, deltoid modulation envelope 250 is further effective for muscle stimulation. Thus, deltoid modulation envelope 250 provides a combination of pain relief and muscle stimulation. In one embodiment, deltoid modulation envelope 250 allows for more robust muscle stimulation without undue pain. While the above advantages have been detailed in relation to deltoid modulation envelope 250, this is not to be limiting in any way, and the advantage may be exhibited by triangular modulation envelope 200 without exceeding the scope of the invention.

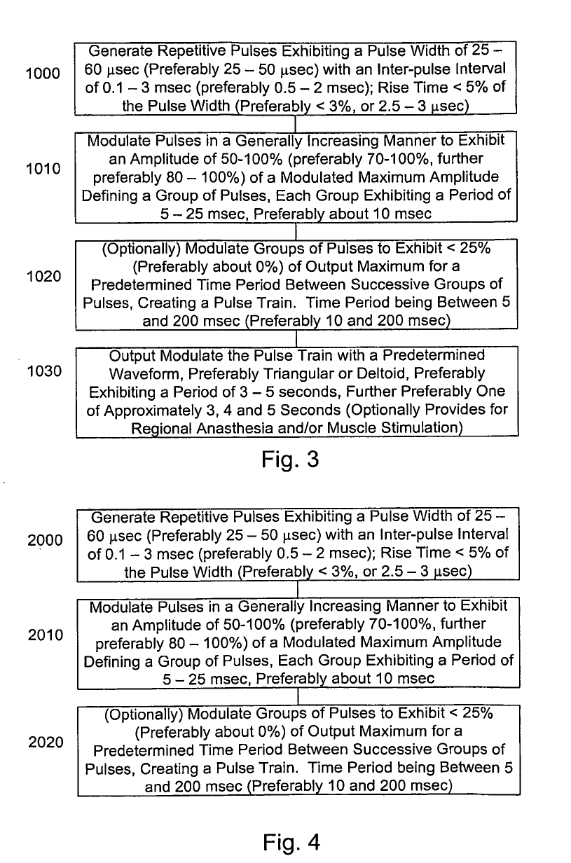

FIG. 3 illustrates a high level flow chart of the operation of modulated pulse generator 10 of FIG. 1 in accordance with certain embodiments of the invention. In stage 1000, repetitive pulses are generated exhibiting an output pulse width of 25-60 microseconds, preferably 25-50 microseconds, with an inter-pulse interval of 0.1-3 milliseconds, preferably 0.5-2 milliseconds. The pulses further exhibit a rise time of no more than 5% of the pulse width. In one embodiment the consistent pulse rise time is no more than 4% of the pulse width. In another embodiment the rise time is no more than 3% of the pulse width and in yet another embodiment the rise time is in the range of 2.5-3 microseconds. In one particular preferred embodiment the repetitive pulses exhibit a pulse width of about 50 microseconds, a consistent pulse rise time of about 1 microsecond and an inter-pulse interval of between 0.1 and 3 milliseconds, and preferably about 1 millisecond.

In stage 1010, the pulses of stage 1000 are modulated in a generally increasing manner to exhibit an amplitude of 50%-100% of a maximum modulated pulse amplitude. The modulated pulses define a group of pulses. In one embodiment each group of pulses exhibit an amplitude of 70%-100% of the maximum modulated pulse amplitude. In another embodiment each group exhibit an amplitude of 80%-100% of the maximum modulated pulse amplitude. Each group of pulses exhibits a period of preferably 5-25 milliseconds, further preferably about 10 milliseconds.

In optional stage 1020, the groups of pulses of stage 1010 are modulated to generate an inter-group period exhibiting an output of <25% of the maximum modulated pulse amplitude. The inter-group period is for a pre-determined time of between 5-200 milliseconds, preferably 10-200 milliseconds. In one embodiment the inter-group period exhibits an output of approximately 0% of the maximum modulated pulse amplitude. The groups of pulses separated by the inter-group period modulation defines a pulse train.

In stage 1030, the pulse train of stage 1020 is modulated with an output repetitive waveform. Preferably the output repetitive waveform is one of a generally triangular and a generally deltoid waveform. In one embodiment the period of the output repetitive waveform is 3-5 seconds, preferably one of approximately 3, 4 and 5 seconds. In one embodiment the pre-determined repetitive waveform exhibits a modulated amplitude of 50%-100% of the maximum output amplitude. In a preferred embodiment the pre-determined repetitive waveform exhibits a modulated amplitude of 70%-100% of the maximum output amplitude.

In an embodiment in which a generally triangular waveform is implemented, preferably the waveform exhibits a substantially linear increase and decrease in amplitude, with the increase and decrease being substantially of the same rate of change. In an embodiment in which a generally deltoid waveform is implemented, preferably the output waveform exhibits a generally linear increase in amplitude for approximately ⅓ of the period, a maximum output pulse amplitude of ⅓ of the period and a generally linear decrease in amplitude for approximately ⅓ of the period. In one particular preferred embodiment the deltoid waveform exhibits a generally increasing linear slope for about 1 second, a generally unchanged maximum output for about 1½ second and a generally decreasing slope for about 1 second for a total period of about 3½ seconds.

The above has been described as sequentially modulating pulses, however this is not meant to be limiting in any way. In particular, the use of a micro-controller or other logic apparatus arranged to directly generate the modulated pulses is specifically included in the scope of the invention.

FIG. 4 illustrates a high level flow chart of the operation of modulated pulse generator 10 of FIG. 1 in accordance with certain embodiments of the invention. In stage 2000, repetitive pulses are generated exhibiting an output pulse width of 25-60 microseconds, preferably 25-50 microseconds, with an inter-pulse interval of 0.1-3 milliseconds, preferably 0.5-2 milliseconds. The pulses further exhibit a rise time of no more than 5% of the pulse width. In one embodiment the consistent pulse rise time is no more than 4% of the pulse width. In another embodiment the rise time is no more than 3% of the pulse width and in yet another embodiment the rise time is in the range of 2.5-3 microseconds. In one particular preferred embodiment the repetitive pulses exhibit a pulse width of about 50 microseconds, a consistent pulse rise time of about 1 microsecond and an inter-pulse interval of between 0.1 and 3 milliseconds, and preferably about 1 millisecond.

In stage 2010, the pulses of stage 1000 are modulated in a generally increasing manner to exhibit an amplitude of 50%-100% of a maximum modulated pulse amplitude. The modulated pulses define a group of pulses. In one embodiment each group of pulses exhibit an amplitude of 70%-100% of the maximum modulated pulse amplitude. In another embodiment each group exhibit an amplitude of 80%-100% of the maximum modulated pulse amplitude. Each group of pulses exhibits a period of preferably 5-25 milliseconds, further preferably about 10 milliseconds. Optionally, each group of pulses further exhibits a leveling off period, which in one embodiment characterizes about 50% of the period. In one particular further embodiment the leveling off is at the maximum amplitude.

In optional stage 2020, the groups of pulses of stage 2010 are modulated to generate an inter-group period exhibiting an output of <25% of the maximum modulated pulse amplitude. The inter-group period is for a pre-determined time of between 5-200 milliseconds, preferably 10-200 milliseconds. In one embodiment the inter-group period exhibits an output of approximately 0% of the maximum modulated pulse amplitude. The groups of pulses separated by the inter-group period modulation defines a pulse train. In one particular embodiment stage 2020 is not implemented.

The above has been described as sequentially modulating pulses, however this is not meant to be limiting in any way. In particular, the use of a micro-controller or other logic apparatus arranged to directly generate the modulated pulses is specifically included in the scope of the invention.

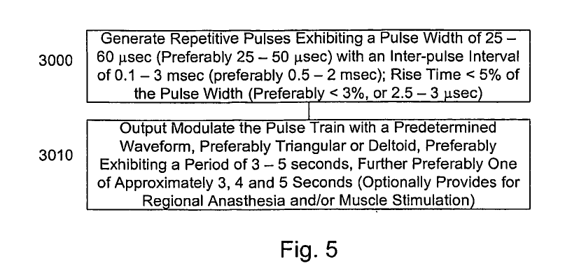

FIG. 5 illustrates a high level flow chart of the operation of modulated pulse generator 10 of FIG. 1 in accordance with certain embodiments of the invention. In stage 3000, repetitive pulses are generated exhibiting an output pulse width of 25-60 microseconds, preferably 25-50 microseconds, with an inter-pulse interval of 0.1-3 milliseconds, preferably 0.5-2 milliseconds. The pulses further exhibit a rise time of no more than 5% of the pulse width. In one embodiment the consistent pulse rise time is no more than 4% of the pulse width. In another embodiment the rise time is no more than 3% of the pulse width and in yet another embodiment the rise time is in the range of 2.5-3 microseconds. In one particular preferred embodiment the repetitive pulses exhibit a pulse width of about 50 microseconds, a consistent pulse rise time of about 1 microsecond and an inter-pulse interval of between 0.1 and 3 milliseconds, and preferably about 1 millisecond.

In stage 3010, the pulses 3000 are modulated with an output repetitive waveform. Preferably the output repetitive waveform is one of a generally triangular and a generally deltoid waveform. In one embodiment the period of the output repetitive waveform is 3-5 seconds, preferably one of approximately 3, 4 and 5 seconds. In one embodiment the pre-determined repetitive waveform exhibits a modulated amplitude of 50%-100% of the maximum output amplitude. In a preferred embodiment the pre-determined repetitive waveform exhibits a modulated amplitude of 70%-100% of the maximum output amplitude.

In an embodiment in which a generally triangular waveform is implemented, preferably the waveform exhibits a substantially linear increase and decrease in amplitude, with the increase and decrease being substantially of the same rate of change. In an embodiment in which a generally deltoid waveform is implemented, preferably the output waveform exhibits a generally linear increase in amplitude for approximately ⅓ of the period, a maximum output pulse amplitude of ⅓ of the period and a generally linear decrease in amplitude for approximately ⅓ of the period. In one particular preferred embodiment the deltoid waveform exhibits a generally increasing linear slope for about 1 second, a generally unchanged maximum output for about 1½ second and a generally decreasing slope for about 1 second for a total period of about 3½ seconds.

The above has been described as sequentially modulating pulses, however this is not meant to be limiting in any way. In particular, the use of a micro-controller or other logic apparatus arranged to directly generate the modulated pulses is specifically included in the scope of the invention.

The above has been described exclusively in connection with the use of an apparatus applying modulated pulses of short duration, however this is not meant to be limiting in any way. In one embodiment the use of one or more of light and heat in combination with the modulated pulses of short duration of the subject invention provides enhanced pain relief. Preferably, the use of light comprises a source of ultraviolet light.

Thus the present embodiments enable an apparatus operative to apply modulated pulses of short duration to the area to be treated. In an exemplary embodiment the area to be treated comprises two points at least 4 centimeters apart generally in consonance with the nerve to be blocked and the modulated pulses comprise constant current pulses of approximately 100 mA each. The pulses exhibit a varying amplitude whose maximum is preferably approximately 100V and are preferably applied to a 16 mm² skin patch.

In a preferred embodiment the pulses are of a constant width, preferably of 25-60 microseconds, even further preferably 25-50 microseconds, with a rise and fall time of no more than 5% of the pulse width, with an inter-pulse interval of between 0.1 and 3 milliseconds. The pulses are further modulated in accordance with various envelopes described herein.

It is appreciated that certain features of the invention, which are, for clarity, described in the context of separate embodiments, may also be provided in combination in a single embodiment. Conversely, various features of the invention which are, for brevity, described in the context of a single embodiment, may also be provided separately or in any suitable sub-combination.

Unless otherwise defined, all technical and scientific terms used herein have the same meanings as are commonly understood by one of ordinary skill in the art to which this invention belongs. Although methods similar or equivalent to those described herein can be used in the practice or testing of the present invention, suitable methods are described herein.

All publications, patent applications, patents, and other references mentioned herein are incorporated by reference in their entirety. In case of conflict, the patent specification, including definitions, will prevail. In addition, the materials, methods, and examples are illustrative only and not intended to be limiting.

It will be appreciated by persons skilled in the art that the present invention is not limited to what has been particularly shown and described hereinabove. Rather the scope of the present invention is defined by the appended claims and includes both combinations and sub-combinations of the various features described hereinabove as well as variations and modifications thereof, which would occur to persons skilled in the art upon reading the foregoing description.

I claim:

1. An apparatus for transcutaneous stimulation comprising:
a pulse generator operative to generate repetitive pulses exhibiting a pulse width of 25-60 microseconds, a consistent output pulse rise time of no more than 5% of the pulse width and an inter-pulse interval of between 0.1 and 3 milliseconds; and at least one of:
an intra-group modulator arranged to modulate said pulses to produce modulated pulses exhibiting an amplitude of between 50% and 100% of a maximum modulated pulse amplitude in a predetermined generally increasing manner, said modulated pulses defining a group of pulses, said intra-group modulator being further operative to modulate said pulses to exhibit an amplitude of no more than 25% of said maximum modulated pulse amplitude for a predetermined inter-group time period between successive groups of pulses thereby creating a pulse train; and
an output modulator operative to output said repetitive pulses exhibiting an amplitude of between 50% and 100% of a maximum output pulse amplitude according to a predetermined repetitive waveform.

2. An apparatus according to claim 1, comprising said intra-group modulator and said output modulator, wherein said output modulator is arranged to modulate said pulse train to output said repetitive pulses.

3. An apparatus according to claim 2, wherein said repetitive pulses exhibit a width of 25-50 microseconds.

4. An apparatus according to any of claims 1-3, wherein said consistent pulse rise time is no more than 4% of the pulse width.

5. An apparatus according to any of claims 1-3, wherein said consistent pulse rise time is no more than 3% of the pulse width.

6. An apparatus according to any of claims 1-5, wherein said inter-pulse interval is between 0.5 and 2 milliseconds.

7. An apparatus according to any of claims 1-6, wherein said modulated pulses exhibit an amplitude of between 70% and 100% of said maximum modulated pulse amplitude.

8. An apparatus according to any of claims 1-6, wherein said modulated pulses exhibit an amplitude of between 80% and 100% of said maximum modulated pulse amplitude.

9. An apparatus according to any of claims 1-8, wherein said intra-group modulator modulates said pulses to exhibit said generally increasing amplitude with a period of 5-25 milliseconds.

10. An apparatus according to any of claims 1-8, wherein said intra-group modulator modulates said pulses to exhibit said generally increasing amplitude with a period of about 10 milliseconds.

11. An apparatus according to any of claims 1-10, wherein said intra-group modulator modulates said pulses to exhibit an amplitude of approximately 0% of said maximum modulated pulse amplitude for said predetermined inter-group time period.

12. An apparatus according to any of claims 1-11, wherein said predetermined inter-group time period is between 5 milliseconds and 200 milliseconds.

13. An apparatus according to any of claims 1-11, wherein said predetermined inter-group time period is between 10 and 200 milliseconds.

14. An apparatus according to any of claims 1-13, wherein said output modulator modulates said pulse train to produce output pulses exhibiting an amplitude of between 70% and 100% of said maximum output pulse amplitude.

15. An apparatus according to any of claims 1-14, wherein said predetermined repetitive waveform is a triangular waveform.

16. An apparatus according to claim 15, wherein said triangular waveform exhibits a period of approximately 3 seconds.

17. An apparatus according to claim 15, wherein said triangular waveform exhibits a period of approximately 4 seconds.

18. An apparatus according to claim 15, wherein said triangular waveform exhibits a period of approximately 5 seconds.

19. An apparatus according to any of claims 15-18, wherein said triangular waveform exhibits a linear increase in modulation and a linear decrease in modulation, said linear increase and said linear decrease exhibiting substantially identical rates of change.

20. An apparatus according to any of claims 1-14, wherein said predetermined repetitive waveform is a deltoid waveform.

21. An apparatus according to claim 20, wherein said deltoid waveform exhibits a period of approximately 3 seconds.

22. An apparatus according to claim 20, wherein said deltoid waveform exhibits a period of approximately 4 seconds.

23. An apparatus according to claim 20, wherein said deltoid waveform exhibits a period of approximately 5 seconds.

24. An apparatus according to any of claims 20-23, wherein said deltoid waveform exhibits a linear increase in output pulse amplitude for approximately ⅓ of the total period, a maximum output pulse amplitude for approximately ⅓ of the total period and a linear decrease in output pulse amplitude for approximately ⅓ of the total period.

25. An apparatus according to claim 20, wherein said deltoid waveform exhibits a linear increase in output pulse amplitude for approximately 1 second, a maximum output pulse amplitude for approximately 1½ seconds and a linear decrease in output pulse amplitude for approximately a second.

26. An apparatus for transcutaneous stimulation comprising:
a pulse generating circuitry arranged to:
generate modulated repetitive pulses exhibiting a pulse width of 25-60 microseconds, a consistent output pulse rise time of no more than 5% of the pulse width and an inter-pulse interval of between 0.1 and 3 milliseconds;
modulate said pulses to produce modulated pulses exhibiting an amplitude of between 50% and 100% of a maximum modulated pulse amplitude in a predetermined generally increasing manner, said modulated pulses defining a group of pulses, said intra-group modulator being further operative to modulate said pulses to exhibit an amplitude of no more than 25% of said maximum modulated pulse amplitude for a predetermined inter-group time period between successive groups of pulses thereby creating a pulse train; and
output modulate said pulse train to exhibit an amplitude of between 50% and 100% of a maximum output pulse amplitude according to a predetermined repetitive waveform.

27. A method of transcutaneous stimulation comprising generating repetitive pulses exhibiting a pulse width of 25-60 microseconds at an interval of between 0.1 and 3 milliseconds, said generated repetitive pulses exhibiting a rise time of no more than 5% of said pulse width, and at least one of:
modulating said repetitive pulses to exhibit an amplitude of between 50% and 100% of a maximum modulated pulse amplitude in a predetermined generally increasing manner, said modulated pulses defining a group of pulses, and further modulating said pulses to exhibit an amplitude of no more than 25% of said maximum modulated pulse amplitude for a predetermined inter-group time period between successive groups of pulses thereby defining a pulse train; and
output modulating said pulses by a predetermined repetitive waveform to produce output pulses exhibiting an amplitude of between 50% and 100% of a maximum output pulse amplitude.

28. A method of transcutaneous stimulation comprising:
generating repetitive pulses exhibiting a pulse width of 25-60 microseconds at an interval of between 0.1 and 3 milliseconds, said generated repetitive pulses exhibiting a rise time of no more than 5% of said pulse width;
modulating said repetitive pulses to exhibit an amplitude of between 50% and 100% of a maximum modulated pulse amplitude in a predetermined generally increasing manner, said modulated pulses defining a group of pulses;
modulating said pulses to exhibit an amplitude of no more than 25% of said maximum modulated pulse amplitude for a predetermined inter-group time period between successive groups of pulses thereby defining a pulse train; and
output modulating said pulse train by a predetermined repetitive waveform to produce output pulses exhibiting an amplitude of between 50% and 100% of a maximum output pulse amplitude.

29. A method according to claim 28, wherein said generated repetitive pulses exhibit a width of 25-50 microseconds.

30. A method according to any of claims 28-29, wherein said consistent pulse rise time is no more than 3% of the pulse width.

31. A method according to any of claims 28-30, wherein said consistent pulse rise time is in the range of 2.5-3 microseconds.

32. A method according to any of claims 28-31, wherein said interval is between 0.5 and 2 milliseconds.

33. A method according to any of claims 28-32, wherein said modulating said repetitive pulses is to exhibit an amplitude of between 70% and 100% of said maximum modulated pulse amplitude.

34. A method according to any of claims 28-32, wherein said modulating said repetitive pulses is to exhibit an amplitude of between 80% and 100% of said maximum modulated pulse amplitude.

35. An apparatus according to any of claims 28-34, wherein generally increasing amplitude exhibits a period of 5-25 milliseconds.

36. An apparatus according to any of claims 28-34, wherein generally increasing amplitude exhibits a period of about 10 milliseconds

37. A method according to any of claims 28-36, wherein said modulating said pulses to exhibit an amplitude of no more than 25% of said maximum modulated pulse amplitude is to exhibit an amplitude of approximately 0% of said maximum modulated pulse amplitude for said predetermined inter-group time period.

38. A method according to any of claims 28-37, wherein said predetermined inter-group time period is between 5 milliseconds and 200 milliseconds.

39. A method according to any of claims 28-37, wherein said predetermined inter-group time period is between 10 and 200 milliseconds.

40. A method according to any of claims 28-39, wherein said output modulating produces output pulses exhibiting an amplitude of between 70% and 100% of said maximum output pulse amplitude.

41. A method according to any of claims 28-40, wherein said predetermined repetitive waveform is a triangular waveform.

42. A method according to claim 41, wherein said triangular waveform exhibits a period of approximately 3 seconds.

43. A method according to claim 41, wherein said triangular waveform exhibits a period of approximately 4 seconds.

44. A method according to claim 41, wherein said triangular waveform exhibits a period of approximately 5 seconds.

45. A method according to any of claims 41-44, wherein said triangular waveform exhibits a linear increase in modulation and a linear decrease in modulation, said linear increase and said linear decrease exhibiting substantially identical rates of change.

46. A method according to any of claims 28-40, wherein said predetermined repetitive waveform is a deltoid waveform.

47. A method according to claim 46, wherein said deltoid waveform exhibits a period of approximately 3 seconds.

48. A method according to claim 46, wherein said deltoid waveform exhibits a period of approximately 4 seconds.

49. A method according to claim 46, wherein said deltoid waveform exhibits a period of approximately 5 seconds.

50. A method according to any of claims 47-49, wherein said deltoid waveform exhibits a linear increase in output pulse amplitude for approximately ⅓ of the total period, a maximum output pulse amplitude for approximately ⅓ of the total period and a linear decrease in output pulse amplitude for approximately ⅓ of the total period.

51. A method according to claim 46, wherein said deltoid waveform exhibits a linear increase in output pulse amplitude for approximately 1 second, a maximum output pulse amplitude for approximately 1½ seconds and a linear decrease in output pulse amplitude for approximately 1 second.

52. A method according to any of claims 28-51, wherein the transcutaneous stimulation provides for regional anesthesia of the area receiving said stimulation.

53. A method according to any of claims 28-51, wherein the transcutaneous stimulation provides for muscle stimulation and pain relief of the area receiving said stimulation.

54. An apparatus for transcutaneous stimulation comprising:
a pulse generator operative to generate repetitive pulses exhibiting a pulse width of 25-60 microseconds, a consistent pulse rise time of no more than 5% of the pulse width and an inter-pulse interval of between 0.5 and 2 milliseconds;
an intra-group modulator arranged to modulate said pulses to exhibit an amplitude of between 70% and 100% of a maximum modulated pulse amplitude in a predetermined generally increasing manner over a period of between 5-25 milliseconds, said modulated pulses defining a group of pulses, said intra-group modulator being further operative to modulate said pulses to exhibit an amplitude of approximately 0% of said maximum modulated pulse amplitude for a predetermined time period of at least 5 milliseconds between successive groups of pulses thereby creating a pulse train; and
an output modulator operative to output modulate said pulse train to exhibit an amplitude of between 70% and 100% of a maximum output pulse amplitude according to a predetermined repetitive waveform.

55. An apparatus for transcutaneous stimulation comprising:
a pulse generator operative to generate repetitive pulses exhibiting a pulse width of about 50 microseconds, a consistent output pulse rise time of about 1 microsecond and an inter-pulse interval of between 0.1 and 3 milliseconds;
an intra-group modulator arranged to receive said pulses and produce modulated pulses exhibiting an amplitude of between 70% and 100% of a maximum modulated pulse amplitude in a predetermined generally increasing manner, over a period of about 10 milliseconds, said modulated pulses defining a group of pulses, said intra-group modulator being further operative to modulate said pulses to exhibit an amplitude of no more than 25% of said maximum modulated pulse amplitude for a predetermined inter-group time period between successive groups of pulses thereby creating a pulse train; and
an output modulator operative to modulate said pulse train to produce output pulses exhibiting an amplitude of between 70% and 100% of a maximum output pulse amplitude according to a predetermined repetitive waveform.

56. A method of muscle stimulation comprising:
generating repetitive pulses exhibiting a pulse width of 25-60 microseconds at an interval of between 0.1 and 3 milliseconds, said generated repetitive pulses exhibiting a rise time of no more than 5% of said pulse width;
modulating said repetitive pulses to exhibit an amplitude of between 50% and 100% of a maximum modulated pulse amplitude in a predetermined generally increasing manner, said modulated pulses defining a group of pulses;
modulating said pulses to exhibit an amplitude of no more than 25% of said maximum modulated pulse amplitude for a predetermined inter-group time period between successive groups of pulses thereby defining a pulse train; and
output modulating said pulse train by a predetermined repetitive deltoid waveform to produce output pulses exhibiting an amplitude of between 50% and 100% of a maximum output pulse amplitude.

57. A method according to claim 56, wherein said deltoid waveform exhibits a linear increase in output pulse amplitude for approximately 1 second, a maximum output pulse amplitude for approximately 1½ seconds and a linear decrease in output pulse amplitude for approximately 1 second.

58. A method according to claim 56 or claim 57, further providing for regional anesthesia.

59. A method according to claim 56 or claim 57, further providing for pain relief.
